(19) United States
(12) Patent Application Publication
(10) Pub. No.: US 2010/0273805 A1
(43) Pub. Date: Oct. 28, 2010

(54) SULPHIDE BRIDGED DERIVATIVES AS MODULATORS OF MGLUR5 733

(75) Inventors: Kenneth Granberg, Molndal (SE); Bjorn Holm, Molndal (SE)

Correspondence Address: BIRCH, STEWART, KOLASCH & BIRCH, LLP, P.O. BOX 747, FALLS CHURCH, VA, 22040-0747, US

(73) Assignee: AstraZeneca AB, Sodertalje (SE)

(21) Appl. No.: 12/764,787

(22) Filed: Apr. 21, 2010

Related U.S. Application Data
(60) Provisional application No. 61/171,950, filed on Apr. 23, 2009.

(51) Int. Cl. A61K 31/501; C07D 401/14; A61K 31/4439; C07D 413/14; A61P 1/04; A61P 29/00; A61P 25/22

(52) U.S. Cl. 514/252.05; 546/268.4; 514/340; 546/269.1; 546/272.1; 544/238; 514/338

(57) ABSTRACT

The present invention is directed to novel compounds, to a process for their preparation, their use in therapy and pharmaceutical compositions comprising the novel compounds.

### FIELD OF THE INVENTION

The present invention is directed to novel compounds, their use in therapy and pharmaceutical compositions comprising said novel compounds.

### BACKGROUND OF THE INVENTION

Glutamate is the major excitatory neurotransmitter in the mammalian central nervous system (CNS). Glutamate produces its effects on central neurons by binding to and thereby activating cell surface receptors. These receptors have been divided into two major classes, the ionotropic and metabotropic glutamate receptors, based on the structural features of the receptor proteins, the means by which the receptors transduce signals into the cell, and pharmacological profiles.

The metabotropic glutamate receptors (mGluRs) are G protein-coupled receptors that activate a variety of intracellular second messenger systems following the binding of glutamate. Activation of mGluRs in intact mammalian neurons elicits one or more of the following responses: activation of phospholipase C; increases in phosphoinositide (PI) hydrolysis; intracellular calcium release; activation of phospholipase D; activation or inhibition of adenyl cyclase; increases or decreases in the formation of cyclic adenosine monophosphate (cAMP); activation of guanylyl cyclase; increases in the formation of cyclic guanosine monophosphate (cGMP); activation of phospholipase A₂; increases in arachidonic acid release; and increases or decreases in the activity of voltage- and ligand-gated ion channels. Schoepp et al., Trends Pharmacol. Sci. 14:13 (1993), Schoepp, Neurochem. Int. 24:439 (1994), Pin et al., Neuropharmacology 34:1 (1995), Bordi and Ugolini, Prog. Neurobiol. 59:55 (1999).

Molecular cloning has identified eight distinct mGluR subtypes, termed mGluR1 through mGluR8. Nakanishi, Neuron 13:1031 (1994), Pin et al., Neuropharmacology 34:1 (1995), Knopfel et al., J. Med. Chem. 38:1417 (1995). Further receptor diversity occurs via expression of alternatively spliced forms of certain mGluR subtypes. Pin et al., PNAS 89:10331 (1992), Minakami et al., BBRC 199:1136 (1994), Joly et al., J. Neurosci. 15:3970 (1995).

Metabotropic glutamate receptor subtypes may be subdivided into three groups, Group I, Group II, and Group III mGluRs, based on amino acid sequence homology, the second messenger systems utilized by the receptors, and by their pharmacological characteristics. Group I mGluR comprises mGluR1, mGluR5 and their alternatively spliced variants. The binding of agonists to these receptors results in the activation of phospholipase C and the to subsequent mobilization of intracellular calcium.

### Neurological, Psychiatric and Pain Disorders

Attempts at elucidating the physiological roles of Group I mGluRs suggest that activation of these receptors elicits neuronal excitation. Various studies have demonstrated that Group I mGluR agonists can produce postsynaptic excitation upon application to neurons in the hippocampus, cerebral cortex, cerebellum, and thalamus, as well as other CNS regions. Evidence indicates that this excitation is due to direct activation of postsynaptic mGluRs, but it also has been suggested that activation of presynaptic mGluRs occurs, resulting in increased neurotransmitter release. Baskys, Trends Pharmacol. Sci. 15:92 (1992), Schoepp, Neurochem. Int. 24:439 (1994), Pin et al., Neuropharmacology 34:1 (1995), Watkins et al., Trends Pharmacol. Sci. 15:33 (1994).

Metabotropic glutamate receptors have been implicated in a number of normal processes in the mammalian CNS. Activation of mGluRs has been shown to be required for induction of hippocampal long-term potentiation and cerebellar long-term depression. Bashir et al., Nature 363:347 (1993), Bortolotto et al., Nature 368:740 (1994), Aiba et al., Cell 79:365 (1994), Aiba et al., Cell 79:377 (1994). A role for mGluR activation in nociception and analgesia also has been demonstrated, Meller et al., Neuroreport 4: 879 (1993), Bordi and Ugolini, Brain Res. 871:223 (1999). In addition, mGluR activation has been suggested to play a modulatory role in a variety of other normal processes including synaptic transmission, neuronal development, apoptotic neuronal death, synaptic plasticity, spatial learning, olfactory memory, central control of cardiac activity, waking, motor control and control of the vestibulo-ocular reflex. Nakanishi, Neuron 13: 1031 (1994), Pin et al., Neuropharmacology 34:1, Knopfel et al., J. Med. Chem. 38:1417 (1995).

Further, Group I metabotropic glutamate receptors and mGluR5 in particular, have been suggested to play roles in a variety of pathophysiological processes and disorders affecting the CNS. These include stroke, head trauma, anoxic and ischemic injuries, hypoglycemia, epilepsy, neurodegenerative disorders such as Alzheimer's disease and pain. Schoepp et al., Trends Pharmacol. Sci. 14:13 (1993), Cunningham et al., Life Sci. 54:135 (1994), Hollman et al., Ann. Rev. Neurosci. 17:31 (1994), Pin et al., Neuropharmacology 34:1 (1995), Knopfel et al., J. Med. Chem. 38:1417 (1995), Spooren et al., Trends Pharmacol. Sci. 22:331 (2001), Gasparini et al. Curr. Opin. Pharmacol. 2:43 (2002), Neugebauer Pain 98:1 (2002). Much of the pathology in these conditions is thought to be due to excessive is glutamate-induced excitation of CNS neurons. Because Group I mGluRs appear to increase glutamate-mediated neuronal excitation via postsynaptic mechanisms and enhanced presynaptic glutamate release, their activation probably contributes to the pathology. Accordingly, selective antagonists of Group I mGluR receptors could be therapeutically beneficial, specifically as neuroprotective agents, analgesics or anticonvulsants.

Recent advances in the elucidation of the neurophysiological roles of metabotropic glutamate receptors generally and Group I in particular, have established these receptors as promising drug targets in the therapy of acute and chronic neurological and psychiatric disorders and chronic and acute pain disorders.

### Gastrointestinal Disorders

The lower esophageal sphincter (LES) is prone to relaxing intermittently. As a consequence, fluid from the stomach can pass into the esophagus since the mechanical barrier is temporarily lost at such times, an event hereinafter referred to as “reflux”.

Gastro-esophageal reflux disease (GERD) is the most prevalent upper gastrointestinal tract disease. Current pharmacotherapy aims at reducing gastric acid secretion, or at neutralizing acid in the esophagus. The major mechanism behind reflux has been considered to depend on a hypotonic lower esophageal sphincter. However, e.g. Holloway & Dent (1990) Gastroenterol. Clin. N. Amer. 19, pp. 517-535, has shown that most reflux episodes occur during transient lower esophageal sphincter relaxations (TLESRs), i.e. relaxations not triggered by swallows. It has also been shown that gastric acid secretion usually is normal in patients with GERD.

The novel compounds according to the present invention are assumed to be useful for the inhibition of transient lower esophageal sphincter relaxations (TLESRs) and thus for the treatment of gastro-esophageal reflux disorder (GERD).

It is well known that certain compounds may cause undesirable effects on cardiac repolarisation in man, observed as a prolongation of the QT interval on electrocardiograms (ECG). In extreme circumstances, this drug-induced prolongation of the QT interval can lead to a type of cardiac arrhythmia called Torsades de Pointes (TdP; Vandenberg et. al. hERG K⁺ channels: friend and foe. Trends Pharmacol Sci 2001; 22: 240-246), leading ultimately to ventricular fibrillation and sudden death. The primary event in this syndrome is inhibition of the rapid component of the delayed rectifying potassium current (IKr) by these compounds. The compounds bind to the aperture-forming alpha sub-units of the channel protein carrying this current—sub-units that are encoded by the human ether-a-go-go-related gene (hERG). Since IKr plays a key role in repolarisation of the cardiac action potential, its inhibition slows repolarisation and this is manifested as a prolongation of the QT interval. Whilst QT interval prolongation is not a safety concern per se, it carries a risk of cardiovascular adverse effects and in a small percentage of people it can lead to TdP and degeneration into ventricular fibrillation.

Generally, compounds of the present invention have low activity against the hERG-encoded potassium channel. In this regard, low activity against hERG in vitro is indicative of low activity in vivo.

It is also desirable for drugs to possess good metabolic stability in order to enhance drug efficacy. Stability against human microsomal metabolism in vitro is indicative of stability towards metabolism in vivo.

Because of their physiological and pathophysiological significance, there is a need for new potent mGluR agonists and antagonists that display a high selectivity for mGluR subtypes, particularly the Group I receptor subtype, most particularly the mGluR5.

The object of the present invention is to provide compounds exhibiting an activity at metabotropic glutamate receptors (mGluRs), especially at the mGluR5 receptor. In particular, the compounds according to the present invention are predominantly peripherally acting, i.e. maintain a low CNS exposure by physical properties and/or active efflux over the blood brain barrier (BBB).

### DESCRIPTION OF THE INVENTION

The present invention relates to a compound of formula I:

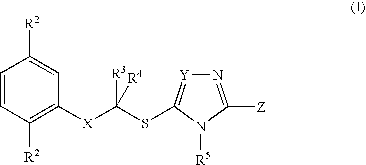

wherein
R¹ is methyl, halogen or cyano;
R² is hydrogen or fluoro;
R³ is hydrogen, C₁-C₃ alkyl or cyclopropyl;
R⁴ is hydrogen, C₁-C₃ alkyl or cyclopropyl;
R⁵ is C₁-C₃ alkyl or cyclopropyl;

### X is

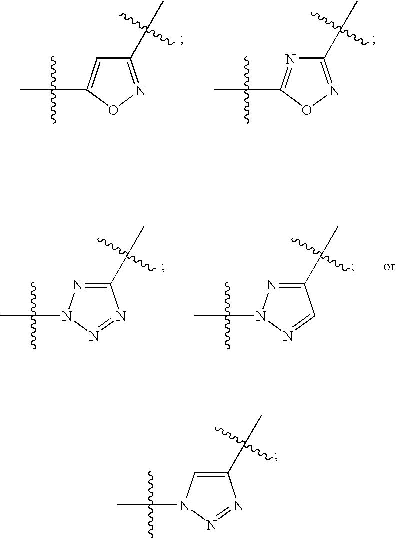

### Y is C or N;

### and Z is

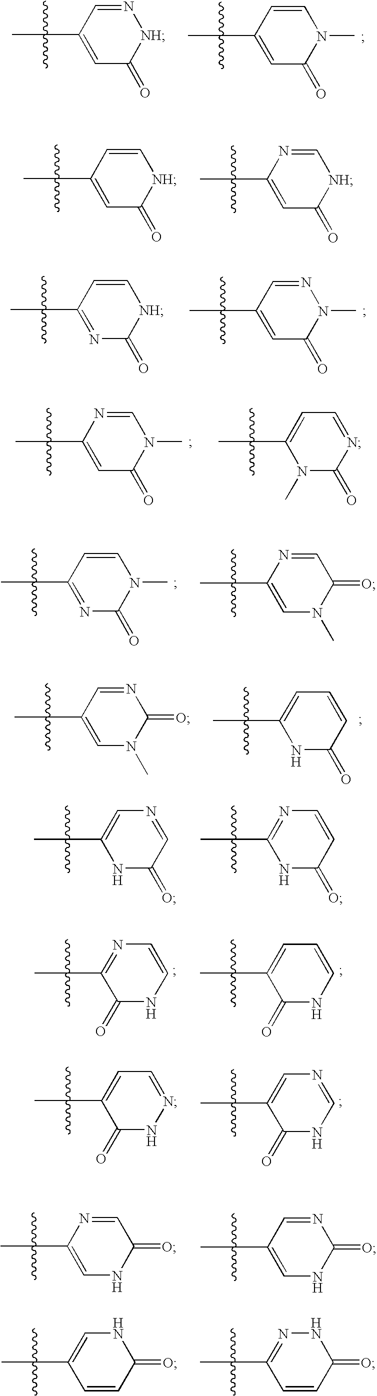

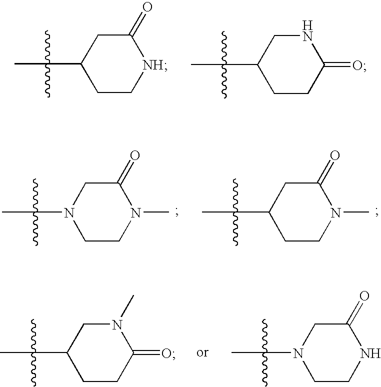

or a pharmaceutically acceptable salt, hydrate, isoform, tautomer or enantiomer thereof.

In one embodiment, R¹ is chloro.

In one embodiment, R¹ is methyl.

In one embodiment, R² is hydrogen.

In one embodiment, R³ is C₁-C₃ alkyl or cyclopropyl and R⁴ is hydrogen.

In one embodiment, the sum of the number of carbon atoms in the substituents R³ and R⁴ is 2 or less.

In one embodiment, R⁵ is methyl.

In one embodiment, X is

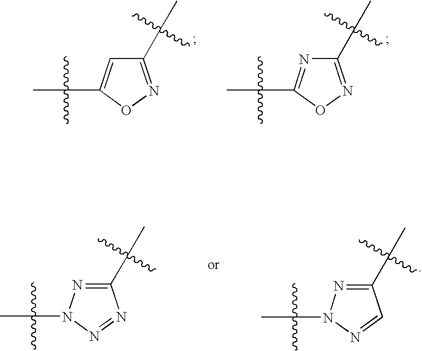

In one embodiment, X is

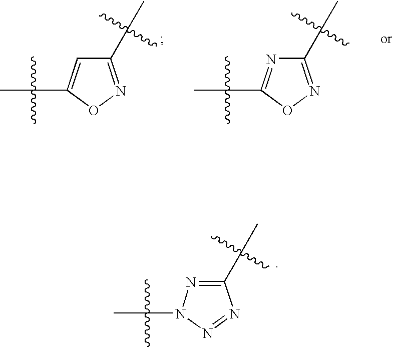

In one embodiment, Y is N.

In one embodiment, Z is

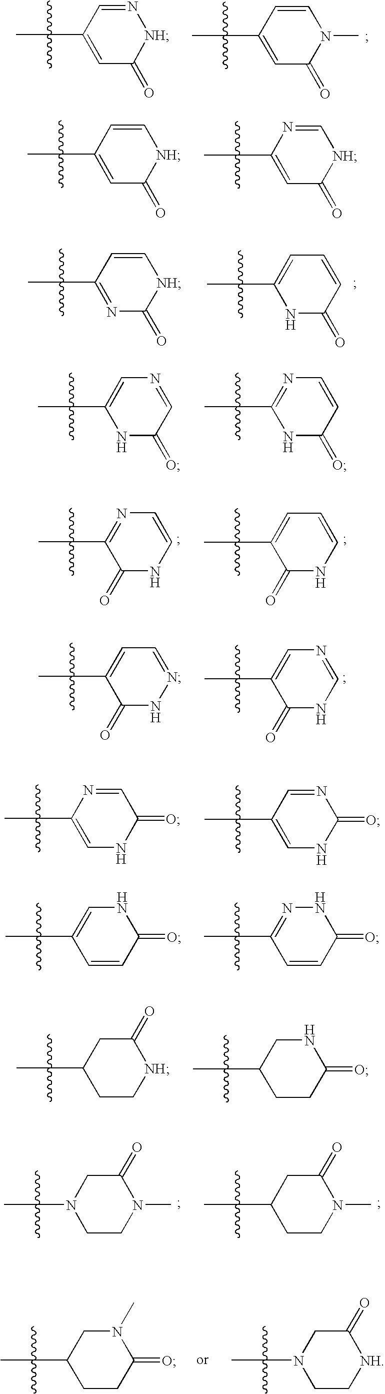

In one embodiment, Z is

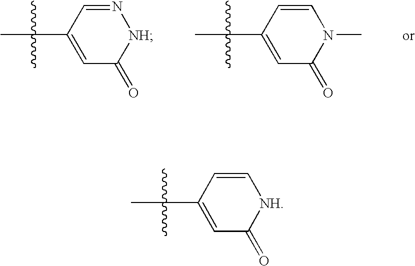

In one embodiment, the orientation of X is such that the compound of formula I has a structure selected from

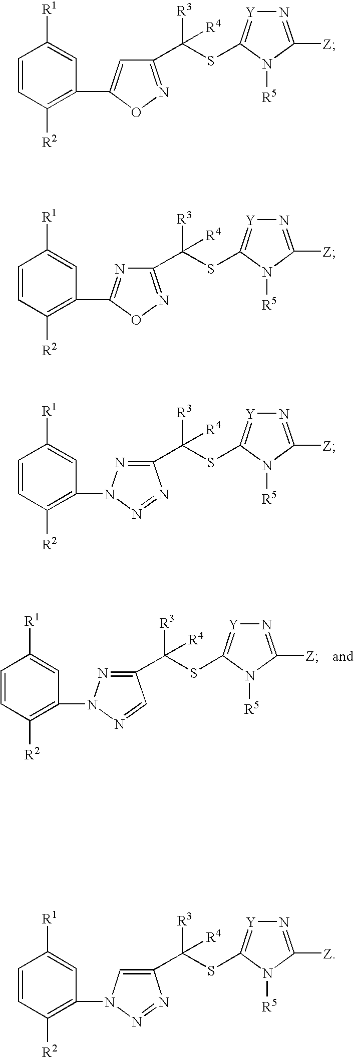

In one embodiment R¹ is methyl or chloro; R² is hydrogen; R³ is methyl; R⁴ is hydrogen; R⁵ is methyl; X is

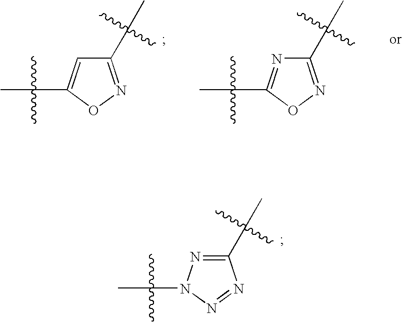

### and Z is

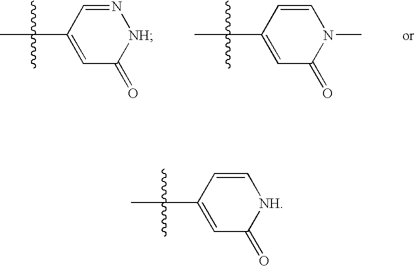

In one embodiment, R⁴ is hydrogen and R³ is C₁-C₃ alkyl or cyclopropyl and the stereochemistry of the compound of formula I is such that R³ projects out of the plane and R⁴ projects into the plane.

In one embodiment, the stereochemistry of the compound of formula I is such that the compound of formula I has the structure

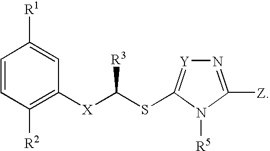

Another embodiment is a pharmaceutical composition comprising as active ingredient a therapeutically effective amount of the compound according to formula I, in association with one or more pharmaceutically acceptable diluents, excipients and/or inert carriers.

Other embodiments, as described in more detail below, relate to a compound according to formula I for use in therapy, in treatment of mGluR5 mediated disorders, in the manufacture of a medicament for the treatment of mGluR5 mediated disorders.

Still other embodiments relate to a method of treatment of mGluR5 mediated disorders, comprising administering to a mammal a therapeutically effective amount of the compound according to formula I.

In another embodiment, there is provided a method for inhibiting activation of mGluR5 receptors, comprising treating a cell containing said receptor with an effective amount of the compound according to formula I.

In a further embodiment a method for the inhibition of transient lower esophageal sphincter relaxations is disclosed wherein an effective amount of the compound according to formula I is administered to a subject in need of such inhibition.

A further embodiment discloses a method for the treatment or prevention of gastroesophageal reflux disease, wherein an effective amount of the compound according to formula I is administered to a subject in need of such treatment or prevention.

Still a further embodiment discloses a method for the treatment or prevention of pain, wherein an effective amount of the compound according to formula I is administered to a subject in need of such treatment or prevention.

Even a further embodiment discloses a method for the treatment or prevention of anxiety, wherein an effective amount of the compound according to formula I is administered to a subject in need of such treatment or prevention.

A further embodiment discloses a method for the treatment or prevention of irritable bowel syndrome (IBS), wherein an effective amount of the compound according to formula I is administered to a subject in need of such treatment or prevention.

The compounds of the present invention are useful in therapy, in particular for the treatment of neurological, psychiatric, pain, and gastrointestinal disorders.

It will also be understood by those of skill in the art that certain compounds of the present invention may exist in solvated, for example hydrated, as well as unsolvated forms. It will further be understood that the present invention encompasses all such solvated forms of the compounds of formula I.

Within the scope of the invention are also salts of the compounds of formula I. Generally, pharmaceutically acceptable salts of compounds of the present invention are obtained using standard procedures well known in the art, for example, by reacting a sufficiently basic compound, for example an amine with a suitable acid, for example, HCl, acetic acid or a methanesulfonic acid to afford a salt with a physiologically acceptable anion. It is also possible to make a corresponding alkali metal (such as sodium, potassium, or lithium) or an alkaline earth metal (such as a calcium) salt by treating a compound of the present invention having a suitably acidic proton, such as a pyridazinone, pyrazinone, pyrimidinone or pyridone with one equivalent of an alkali metal or alkaline earth metal hydroxide or alkoxide (such as the ethoxide or methoxide), followed by conventional purification techniques.

In one embodiment of the present invention, the compound of formula I may be converted to a pharmaceutically acceptable salt or solvate thereof, particularly, an acid addition salt such as a acetate, fumarate, maleate, tartrate, citrate, hydrochloride, hydrobromide, sulphate, phosphate, methanesulphonate or p-toluenesulphonate.

The general terms used in the definition of formula I have the following meanings:

Halogen as used herein is selected from chlorine, fluorine, bromine or iodine.

C₁-C₃ alkyl is a straight or branched alkyl group, having from 1 to 3 carbon atoms, for example methyl, ethyl, n-propyl or isopropyl.

All chemical names were generated using ChemDraw StructureToName v9.07.

In formula I above, X may be present in any of the two possible orientations.

### Pharmaceutical Composition

The compounds of the present invention may be formulated into conventional pharmaceutical compositions comprising a compound of formula I, or a pharmaceutically acceptable salt or solvate thereof, in association with a pharmaceutically acceptable carrier or excipient. The pharmaceutically acceptable carriers can be either solid or liquid. Solid form preparations include, but are not limited to, powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents. A solid carrier can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized moulds and allowed to cool and solidify.

Suitable carriers include, but are not limited to, magnesium carbonate, magnesium stearate, talc, lactose, sugar, pectin, dextrin, starch, tragacanth, methyl cellulose, sodium carboxymethyl cellulose, low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art. Exemplary compositions intended for oral use may contain one or more coloring, sweetening, flavoring and/or preservative agents.

Depending on the mode of administration, the pharmaceutical composition will include from about 0.05% w (percent by weight) to about 99% w, or from about 0.10% w to 50% w, of a compound of the invention, all percentages by weight being based on the total weight of the composition.

A therapeutically effective amount for the practice of the present invention can be determined by one of ordinary skill in the art using known criteria including the age, weight and response of the individual patient, and interpreted within the context of the disease which is being treated or which is being prevented.

The pharmaceutical compositions of this invention will normally be administered to humans so that, for example, a daily dose of 0.01 to 25 mg/kg body weight (and preferably of 0.1 to 5 mg/kg body weight) is received. This daily dose may be given in divided doses as necessary, the precise amount of the compound received and the route of administration depending on the weight, age and sex of the patient being treated and on the particular disease condition being treated according to principles known in the art.

Typically unit dosage forms will contain about 1 mg to 500 mg of a compound of this invention. For example a tablet or capsule for oral administration may conveniently contain up to 250 mg (and typically 5 to 100 mg) of a compound of the formula I or a pharmaceutically acceptable salt thereof. In another example, for administration by inhalation, a compound of the formula I or a pharmaceutically acceptable salt thereof may be administered in a daily dosage range of 5 to 100 mg, in a single dose or divided into two to four daily doses. In a further example, for administration by intravenous or intramuscular injection or infusion, a sterile solution or suspension containing up to 10% w/w (and typically 5% w/w) of a compound of the formula I or a pharmaceutically acceptable salt thereof may be used.

### Medical Use

The compounds according to the present invention are useful in the treatment of conditions associated with excitatory activation of mGluR5 and for inhibiting neuronal damage caused by excitatory activation of mGluR5. The compounds may be used to produce an inhibitory effect of mGluR5 in mammals, including man.

The Group I mGluR receptors including mGluR5 are highly expressed in the central and peripheral nervous system and in other tissues. Thus, it is expected that the compounds of is the invention are well suited for the treatment of mGluR5 mediated disorders such as acute and chronic neurological and psychiatric disorders, gastrointestinal disorders, and chronic and acute pain disorders.

The invention relates to compounds of formula I, as defined herein before, for use in therapy.

The invention relates to compounds of formula I, as defined herein before, for use in treatment of mGluR5 mediated disorders.

The invention relates to compounds of formula I, as defined herein before, for use in treatment of Alzheimer's disease senile dementia, AIDS-induced dementia, Parkinson's disease, Parkinson disease-Ldopa induced dyskinesia, amyotrophic lateral sclerosis, Huntington's Chorea, migraine, epilepsy, schizophrenia, depression, anxiety, acute anxiety, opthalmological disorders such as retinopathies, diabetic retinopathies, glaucoma, auditory neuropathic disorders such as tinnitus, chemotherapy induced neuropathies, post-herpetic neuralgia and trigeminal neuralgia, tolerance, dependency, Fragile X, autism, mental retardation, schizophrenia and Down's Syndrome.

The invention relates to compounds of formula I, as defined above, for use in treatment of pain related to migraine, inflammatory pain, neuropathic pain disorders such as diabetic neuropathies, arthritis and rheumatoid diseases, low back pain, post-operative pain and pain associated with various conditions including cancer, angina, renal or biliary colic, menstruation, migraine and gout.

The invention relates to compounds of formula I as defined herein before, for use in treatment of stroke, head trauma, anoxic and ischemic injuries, hypoglycemia, cardiovascular diseases and epilepsy.

The present invention relates also to the use of a compound of formula I as defined herein before, in the manufacture of a medicament for the treatment of mGluR Group I receptor-mediated disorders and any disorder listed above.

One embodiment of the invention relates to the use of a compound according to formula I in the treatment of gastrointestinal disorders.

Another embodiment of the invention relates a compound of formula I for the inhibition of transient lower esophageal sphincter relaxations, for the treatment of GERD, for the prevention of gastroesophageal reflux, for the treatment of regurgitation, for the treatment of asthma, for the treatment of laryngitis, for the treatment of lung disease, for the management of failure to thrive, for the treatment of irritable bowel syndrome (IBS) and for the treatment of functional dyspepsia (FD). Another embodiment of the invention relates a compound of formula I for the treatment of cough and Barret's Esophagus.

Another embodiment of the invention relates to the use of a compound of formula I for the manufacture of a medicament for inhibition of transient lower esophageal sphincter relaxations, for the treatment of GERD, for the prevention of gastroesophageal reflux, for the treatment regurgitation, for the treatment of asthma, for the treatment of laryngitis, for the treatment of lung disease, for the management of failure to thrive, for the treatment of irritable bowel syndrome (IBS) and for the treatment of functional dyspepsia (FD).

Another embodiment of the invention relates to the use of a compound of formula I for the manufacture of a medicament for the treatment of cough and Barret's Esophagus.

Another embodiment of the present invention relates to the use of a compound of formula I for the treatment of overactive bladder or urinary incontinence.

The wording “TLESR”, transient lower esophageal sphincter relaxations, is herein defined in accordance with Mittal, R. K, Holloway, R. H., Penagini, R., Blackshaw, L. A., Dent, J., 1995; Transient lower esophageal sphincter relaxation. Gastroenterology 109, pp. 601-610.

The wording “reflux” is herein defined as fluid from the stomach being able to pass into the esophagus, since the mechanical barrier is temporarily lost at such times.

The wording “GERD”, gastro-esophageal reflux disease, is herein defined in accordance with van Heerwarden, M. A., Smout A. J. P. M., 2000; Diagnosis of reflux disease. Baillière's Clin. Gastroenterol. 14, pp. 759-774.

The compounds of formula I above are useful for the treatment or prevention of obesity or overweight, (e.g., promotion of weight loss and maintenance of weight loss), prevention or reversal of weight gain (e.g., rebound, medication-induced or subsequent to cessation of smoking), for modulation of appetite and/or satiety, eating disorders (e.g. binge eating, anorexia, bulimia and compulsive) and cravings (for drugs, tobacco, alcohol, any appetizing macronutrients or non-essential food items).

The invention also provides a method of treatment of mGluR5-mediated disorders and any disorder listed above, in a patient suffering from, or at risk of, said condition, which comprises administering to the patient an effective amount of a compound of formula I, as herein before defined.

The dose required for the therapeutic or preventive treatment of a particular disorder will necessarily be varied depending on the host treated, the route of administration and the severity of the illness being treated.

In the context of the present specification, the term “therapy” and “treatment” includes prevention or prophylaxis, unless there are specific indications to the contrary. The terms “therapeutic” and “therapeutically” should be construed accordingly.

In this specification, unless stated otherwise, the term “antagonist” and “inhibitor” shall mean a compound that by any means, partly or completely, blocks the transduction pathway leading to the production of a response by the ligand.

The term “disorder”, unless stated otherwise, means any condition and disease associated with metabotropic glutamate receptor activity.

One embodiment of the present invention is a combination of a compound of formula I and an acid secretion inhibiting agent. A “combination” according to the invention may be present as a “fix combination” or as a “kit of parts combination”. A “fix combination” is defined as a combination wherein the (i) at least one acid secretion inhibiting agent; and (ii) at least one compound of formula I are present in one unit. A “kit of parts combination” is defined as a combination wherein the (i) at least one acid secretion inhibiting agent; and (ii) at least one compound of formula I are present in more than one unit. The components of the “kit of parts combination” may be administered simultaneously, sequentially or separately. The molar ratio of the acid secretion inhibiting agent to the compound of formula I used according to the invention in within the range of from 1:100 to 100:1, such as from 1:50 to 50:1 or from 1:20 to 20:1 or from 1:10 to 10:1. The two drugs may be administered separately in the same ratio. Examples of acid secretion inhibiting agents are H2 blocking agents, such as cimetidine, ranitidine; as well as proton pump inhibitors such as pyridinylmethylsulfinyl benzimidazoles such as omeprazole, esomeprazole, lansoprazole, pantoprazole, rabeprazole or related substances such as leminoprazole.

### Non-Medical Use

In addition to their use in therapeutic medicine, the compounds of formula I, as well as salts and hydrates of such compounds, are useful as pharmacological tools in the development and standardisation of in vitro and in vivo test systems for the evaluation of the effects of inhibitors of mGluR related activity in laboratory animals such as cats, dogs, rabbits, monkeys, rats and mice, as part of the search for new therapeutic agents.

### Methods of Preparation

Another aspect of the present invention provides processes for preparing compounds of formula I, or salts or hydrates thereof. Processes for the preparation of the compounds in the present invention are described herein.

### ABBREVIATIONS

### ACN Acetonitrile

### DCM Dichloromethane

### DMF N,N-Dimethylformamide

### DMSO Dimethylsulfoxide

### Et Ethyl

EtOAc Ethyl acetate

### EtOH Ethanol

h hour(s)
HOAc Acetic acid

### HPLC High Performance Liquid Chromatography

### HRMS High Resolution Mass Spectrometry

### MeOH Methanol

### MeTHF 2-Methyltetrahydrofuran

min minute(s)
MTBE Methyl tert-butyl ether

### NMR Nuclear Magnetic Resonance

### TEA Triethylamine

### THF Tetrahydrofuran

R.t. Room temperature

Throughout the following description of such processes it is to be understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Green's Protective Groups in Organic Synthesis”, 4^{th} Edition, P. G. M. Wuts, T. W. Green, Wiley-Interscience, New York, (2006). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to the one skilled in the art of organic synthesis. Examples of transformations are given below, and it is to be understood that the described transformations are not limited only to the generic groups or substituents for which the transformations are exemplified. References and descriptions on other suitable transformations are given in “Comprehensive Organic Transformations—A Guide to Functional Group Preparations”, 2^{nd} Edition R. C. Larock, VHC Publishers, Inc. (1999). References and descriptions of other suitable reactions are described in textbooks of organic chemistry, for example, “Advanced Organic Chemistry: Reactions, Mechanisms, and Structure”, 6^{th} Edition, Michael B. Smith and Jerry March, McGraw Hill (2007) or, “Organic Synthesis”, 2^{nd} Edition, Michael B. Smith, McGraw Hill, (2001). Techniques for purification of intermediates and final products include for example, straight and reversed phase chromatography on column or rotating plate, recrystallisation, distillation and liquid-liquid or solid-liquid extraction, which will be readily understood by the one skilled in the art. The definitions of substituents and groups are as in formula I except where defined differently. The term r.t. shall mean, unless otherwise specified, a temperature between 16 and 25° C.

The term “reflux” shall mean, unless otherwise stated, in reference to an employed solvent a temperature at or above the boiling point of named solvent.

### Preparation of Intermediates

The intermediates provided in synthetic paths given below, are useful for further preparation of compounds of formula I. Other starting materials are either commercially available or can be prepared via methods described in the literature. The synthetic pathways described below are non-limiting examples of preparations that can be used. One of skill in the art would understand other pathways might be used.

### Formation of Compounds of Formula V

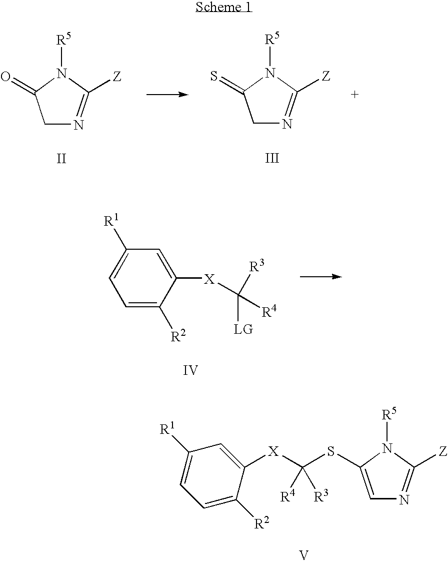

A compound of formula V may be prepared through nucleophilic substitution of compounds of formula IV with compounds of formula III. Compounds of formula III may be prepared by reaction of their oxo analogues II using Lawesson's reagent or P₄S₁₀ at elevated temperature. Synthesis of compounds of formula II has been described by Takeuchi, H., Hagiwara, S., Eguchi, S., Tetrahedron (1989); 45; 6375-6386.

### Formation of Compounds of Formula VI

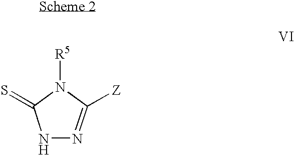

Synthesis of compounds of formula VI has been described in WO2008/041075.

### Formation of Compounds of Formula VII

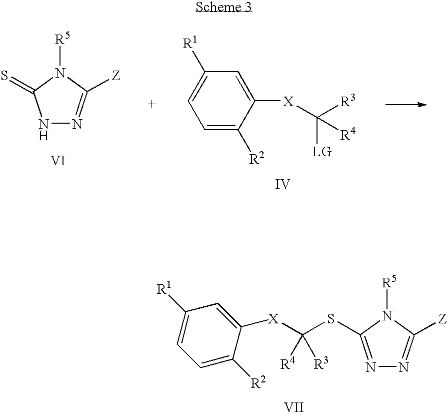

A compound of formula VII may be prepared through nucleophilic substitution of compounds of formula IV with compounds of formula VI in a suitable aprotic solvent, for example DMF or DMSO and in the presence of a suitable base, for example DIPEA or Et₃N at temperature from 50 to 150° C. The leaving group, LG, may include e.g. halides and sulphonates such as mesylate and tosylate.

### General Synthesis of Alcohols of Formula X

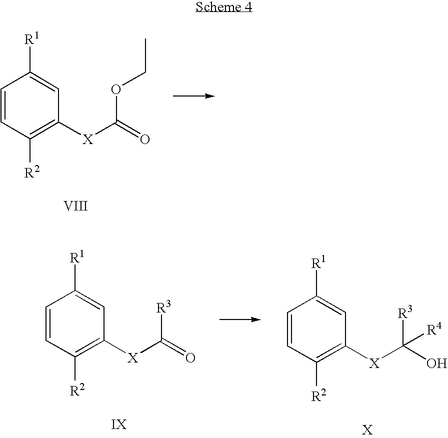

Esters of formula VIII can be transformed into ketones of formula IX by use of the appropriate alkylgrignard reagent (R³—MgBr) in the presence of TEA in a solvent such as THF, MeTHF, toluene or mixtures thereof. (Kikkawa et. al., Synthesis (1980), 11, 877-80). Enantiomerically enriched secondary alcohols of formula X (in which R³ or R⁴═H) can be obtained by reduction with borane-dimethylsulfide in the presence of a suitable chiral catalyst (e.g. (R)-1-methyl-3,3-diphenylhexahydropyrrolo[1,2-c][1,3,2]oxazaborole). Further examples of methods of preparation of alcohols of formula X can be found in the literature (e.g. WO2005/080356 and WO2008/041075).

### Formation of Compounds of Formula XIV

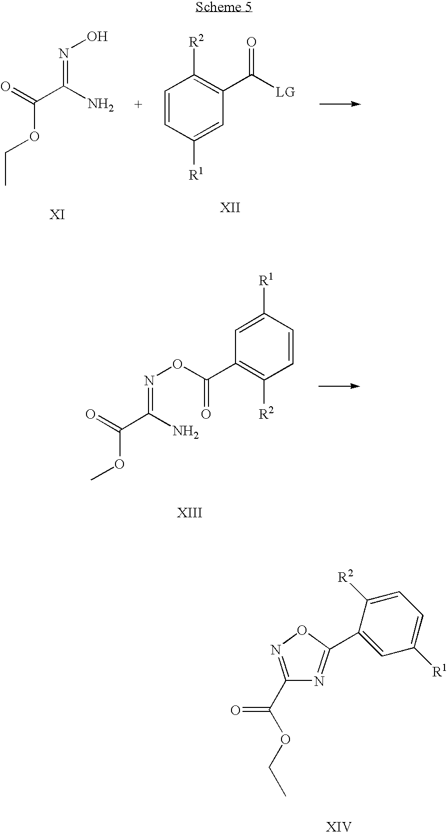

A compound of XIV can be prepared in two consecutive steps via an isolated intermediate of formula XIII. The formation of the intermediate of formula XIII through nucleophilic substitution of compounds of formula XII with compounds of formula XI in a suitable aprotic solvent, for example DMF, DCM, THF or MTBE with an suitable base, for example DIPEA or Et₃N. The cyclization of compounds of formula XIII to form an oxadiazole may be carried out on the ester at temperature from 100° C. to 150° C. in a in a suitable aprotic solvent, for example xylenes or toluene. A catalytic amount of acid can be added if needed (e.g. H₂SO₄).

### EXAMPLES

The invention will now be illustrated by the following non-limiting examples.

### General Methods

The ¹H NMR spectra were recorded either on Varian INOVA spectrometers operating at 400, 500 and 600 MHz for ¹H NMR respectively, using TMS or the residual solvent signal as reference, in deuterated chloroform as solvent unless otherwise indicated. All reported chemical shifts are in ppm on the delta-scale, and the fine splitting of the signals as appearing in the recordings (s: singlet, br s: broad singlet, d: doublet, t: triplet, q: quartet, m: multiplet). Automated flash chromatography was performed on Biotage® KP-SIL columns. Preparative reversed phase HPLC was performed on XBridge C18 columns (10 μm, 250×50 mm or 250×20 mm) with a flow of 100 mL/min using gradients of ACN in buffers consisting of either H₂O/ACN/NH₃ (95:5:0.2), H₂O/ACN/HCOOH (95:5:0.2) or H₂O/ACN/HOAc (95:5:0.2).

The following methods were used for HPLC on chiral stationary phases:

Method A: The isomers were separated on a Chiralcel OJ (250×4.6 mm, 5 μm) HPLC column eluted with EtOH/TEA (100:0.1) at a flow rate of 1 mL/min and detected at 267 nm.
Method B: The isomers were separated on a ChiralPak AD (250×4.6 mm, 5 μm) HPLC column eluted with Heptane/IPA (40:60) at a flow rate of 1 mL/min and detected at 256 nm.
Method C: The isomers were separated on a ReproSil (250×4.6 mm, 8 μm) HPLC column eluted with EtOH/TEA (100:0.1) at a flow rate of 1 mL/min and detected at 267 nm.
Method D: The isomers were separated on a ChiralPak AS (250×4.6 mm, 5 μm) HPLC column eluted with Heptane/EtOH/TEA (30:70:0.1) at a flow rate of 1 mL/min and detected at 254 nm.
Method E: The isomers were separated on a ChiralPak AS (250×4.6 mm, 5 μm) HPLC column eluted with Heptane/EtOH/TEA (40:60:0.1) at a flow rate of 1 mL/min and detected at 254 nm.
Method F: The isomers were separated on a ChiralPak AS (250×4.6 mm, 5 μm) HPLC column eluted with Heptane/EtOH/TEA (20:80:0.1) at a flow rate of 1 mL/min and detected at 254 nm.
Method G: The isomers were separated on a ChiralPak AS (250×4.6 mm, 5 μm) HPLC column eluted with Heptane/EtOH/TEA (85:15:0.1) at a flow rate of 1 mL/min and detected at 275 nm.

### Example 1

### (E)-Ethyl 2-amino-2-(3-methylbenzoyloxyimino)acetate

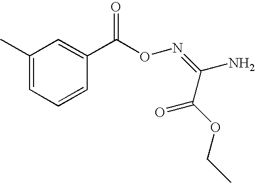

(E)-Ethyl 2-amino-2-(hydroxyimino)acetate (25.6 g, 193 mmol) was added to MTBE (200 mL) and DCM (500 mL). The mixture was cooled to 0° C. and TEA (32.5 mL, 232 mmol) was added. 3-Methylbenzoyl chloride (30.6 g, 198 mmol) in MTBE (20 mL) was added to the mixture dropwise (15 min) and the mixture was stirred at r.t. for 3 h, concentrated. NaHCO₃ (sat, aq) was added to the solid mixture. The solid was filtered off and washed with H₂O and dried under vacuum to give crude title compound (38.4 g, 78%) that was used directly in the next step.

### Example 2

### Ethyl 5-m-tolyl-1,2,4-oxadiazole-3-carboxylate

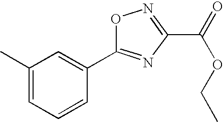

Crude (E)-ethyl 2-amino-2-(3-methylbenzoyloxyimino)acetate (23.8 g, 95.1 mmol) was added to xylenes (200 mL) and H₂SO₄ (0.05 mL) was added and the mixture heated to 130° C. over night. The mixture was concentrated and the residue was purified via Biotage heptane/EtOAc to give the title compound (17.5 g, 79%).

¹H NMR (400 MHz, CDCl₃) δ 1.48 (t, 3H), 2.45 (s, 3H), 4.55 (q, 2H), 7.42-7.47 (m, 2H), 7.99-8.04 (m, 1H), 8.05-8.07 (m, 1H).

### Example 3

### 1-(2-m-Tolyl-2H-tetrazol-5-yl)ethanone

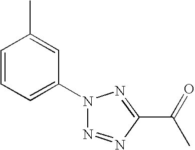

### Step A

m-Toluidine (6.86 g, 63.4 mmol) was dissolved in EtOH (20 mL). Water (7 mL) and 37% HCl (13 mL, 158 mmol) was added and the solution was cooled to −5° C. A solution of sodium nitrite (4.96 g, 69.7 mmol) in water (14 mL) was added to the reaction mixture while keeping the reaction temperature below 5° C. A solution of sodium acetate (15.60 g, 190.1 mmol) in water (31 mL), was added while keeping the reaction temperature below 0° C. Ethyl 2-chloroacetoacetate (10.87 g, 63.38 mmol) was added and the reaction mixture was stirred overnight at 27° C. MeTHF (27 mL) was added and the temperature was adjusted to 40° C. The aqueous layer was discarded and the solution of (Z)-ethyl 2-chloro-2-(2-m-tolylhydrazono)acetate was used as such in the next step.

### Step B

Ammonium hydroxide (25% solution in water, 29 mL, 393 mmol) was added at 0° C. whereupon the mixture was warmed to 17° C. for 2 h. The aqueous layer was discarded and MeTHF (14 mL) was added. The same volume solvent was distilled off at 50° C. under reduced pressure. The procedure was repeated using 21 mL MeTHF and the solution was concentrated to 27 mL. The temperature was adjusted to 30° C. after which n-heptane (27 mL) was added and the solution was cooled to 5° C. during 4 h. Another portion of n-heptane (27 mL) was added to the resulting slurry. The product was isolated by filtration, washed with n-heptane (27 mL) and was dried at 40° C. under reduced pressure giving (Z)-ethyl 2-amino-2-(2-m-tolylhydrazono)acetate, 11.2 g as a brown yellow powder in 72% yield over two steps.

### Step C

A solution of sodium nitrite (3.94 g, 46.2 mmol) in water (22 mL) was added dropwise over 1 h to a solution of (Z)-ethyl 2-amino-2-(2-m-tolylhydrazono)acetate (11.19 g, 48.70 mmol) in MeTHF (112 mL) and HOAc (11 ml, 185 mmol) held at 70° C. The solution was cooled to 35° C. and the aqueous phase was discarded. The organic phase was washed with water (22 mL) followed by potassium carbonate (15.95 g, 115.44 mmol) dissolved in water (45 mL). The organic phase was concentrated by 50% under reduced pressure at 50° C. The resulting solution containing ethyl 2-m-tolyl-2H-tetrazole-5-carboxylate was used as such in the following step.

### Step D

To methyl magnesium bromide 1.4 M in toluene/THF (3:1) (59.4 mL, 83.1 mmol) was added TEA (35 mL, 249 mmol) at r.t. The mixture was cooled to −20° C. and was added dropwise while keeping the inner temperature below −10° C. to the above solution of ethyl 2-m-tolyl-2H-tetrazole-5-carboxylate. The reaction mixture was quenched by adding the mixture to HOAc (26 mL, 462 mmol) in MeTHF (45 mL) while keeping the reaction temperature below 0° C. After complete addition, the mixture was warmed to 50° C. and the aqueous phase was discarded. The organic phase was washed with water (45 mL) followed by potassium carbonate (10.2 g, 73.9 mmol) dissolved in water (45 mL). The organic phase was concentrated to 20 mL under reduced pressure at 50° C. and isopropanol (60 mL) was added and then cooled to 5° C. over 4 h. The product was isolated by filtration, washed with cooled isopropanol (22 mL) and dried under reduced pressure at 40° C. to give the title compound (5.46 g, 57%).

¹H NMR (400 MHz, CDCl₃) δ 2.49 (s, 3H), 2.85 (s, 3H), 7.36 (d, 1H), 7.47 (t, 1H), 8.00 (d, 1H), 8.03 (s, 1H).

### Example 4.1

### 1-(5-m-Tolylisoxazol-3-yl)ethanone

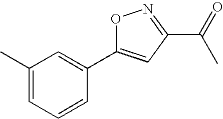

Ethyl 5-m-tolylisoxazole-3-carboxylate (29.0 g, 125 mmol) was dissolved in toluene (400 mL) and cooled to 0° C., a mixture of TEA (87 mL, 627 mmol), methylmagnesium bromide (29.9 g, 25 mmol, 1.4 M in toluene/THF (75:25) and THF (30 mL) was added dropwise over 45 min. The mixture was stirred at 0° C. over night before it was quenched with 2 M HCl over 4 h (pH ˜3). MTBE (300 mL) was added and the mixture was stirred vigorously for 20 min. The aqueous layer was removed and the organic phase was washed with brine (200 mL). The organic phase was dried over magnesium sulfate, filtered and concentrated under reduced pressure. The residue was purified by automated flash chromatography using a gradient of DCM in heptane as eluent to give the title compound (11.7 g, 46%).

¹H NMR (400 MHz, DMSO-d6) δ 2.39 (s, 3H), 2.62 (s, 3H), 7.34-7.39 (m, 2H), 7.41-7.47 (m, 1H), 7.72-7.81 (m, 2H).

In a similar manner the following compound was synthesized:

| Example | Structure | Name | Yield |
|---|---|---|---|
| 4.2 | 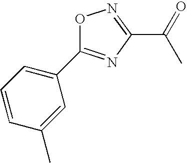 | 1-(5-m-Tolyl-1,2, 4-oxadiazol-3- yl)ethanone | 818 mg, 23% |

| | |
|---|---|
| ¹H NMR | (600 MHz, DMSO-d6) δ 2.42 (s, 3H), 2.65 (s, 3H), 7.49-7.58 |
| | (m, 2H), 7.95 (d, 1H), 7.98 (s, 1H) |

### Example 5.1

### (S)-1-(5-m-Tolylisoxazol-3-yl)ethanol

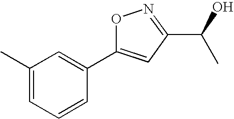

(R)-1-Methyl-3,3-diphenylhexahydropyrrolo[1,2-c][1,3,2]oxazaborole in Toluene (0.90 mL, 0.89 mmol) was diluted with THF (1 mL) and borane dimethylsulfide complex (0.566 mL, 5.96 mmol) was added dropwise. 1-(5-m-tolylisoxazol-3-yl)ethanone (1.5 g, 7.45 mmol) was dissolved in THF (20 mL) and added dropwise during 3 h. After addition the mixture was stirred for 20 min and then cooled to 0° C. MeOH (5 mL) was added dropwise (30 min) and the mixture was stirred for 20 min Aqueous 2 M HCl (15 mL, 30 mmol) was added dropwise over 40 min and the mixture was stirred for 20 min Toluene (100 mL) was added and the mixture was allowed to reach r.t over 20 min and the layers were separated. The organic layer was concentrated and the residue was purified by automated flash chromatography using a gradient of MeOH in DCM as eluent to give the title compound (1.27 g, 84%).

¹H NMR (400 MHz, DMSO-d6) δ 1.42 (d, 3H), 2.38 (s, 3H), 4.78-4.88 (m, 1H), 5.58 (d, 1H), 6.97 (s, 1H), 7.27-7.33 (m, 1H), 7.37-7.44 (m, 1H), 7.62-7.72 (m, 2H).

In a similar manner the following compounds were synthesized:

| Example | Structure | Name | Yield |
|---|---|---|---|
| 5.2 | 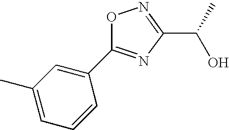 | (S)-1-(5-m-Tolyl-1,2,4-oxadiazol- 3-yl)ethanol | 312 mg, 60% |
| ¹H NMR | (400 MHz, DMSO-d6) δ 1.46 (d, 3H), 2.40 (s, 3H), 4.88 (s, 1H), 5.75 (d, | | |
| | 1H), 7.50 (dd, 2H), 7.88 (dd 1H), 7.92 (s, 1H) | | |
| 5.3 | 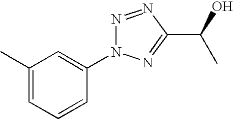 | (S)-1-(2-m-tolyl-2H-tetrazol-5- yl)ethanol | 19.6 g, 97% |

| | |
|---|---|
| ¹H NMR | (400 MHz, DMSO-d6) δ 1.55 (d, 3H), 2.43 (s, 3H), 5.04-5.12 (m, 1H), |
| | 5.76 (d, 1H), 7.37-7.42 (m, 1H), 7.49-7.57 (m, 1H), 7.81-7.92 (m, 2H) |

### Example 6.1

### (S)-1-(5-m-Tolylisoxazol-3-yl)ethyl methanesulfonate

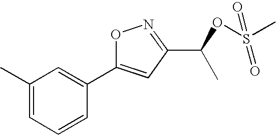

(S)-1-(5-m-Tolylisoxazol-3-yl)ethanol (1.27 g, 6.25 mmol) was dissolved in DCM (25 mL) and methanesulfonyl chloride (0.822 mL, 10.6 mmol) was added. TEA (1.73 mL, 12.5 mmol) was added and the mixture stirred for 20 min. The mixture was diluted with DCM (50 mL) and H₂O (100 mL), the H₂O layer was extracted with DCM and the combined organic layers were dried over Na₂SO₄ and concentrated to give the title compound (1.76 g, 100%).

¹H NMR (600 MHz, DMSO-d6) δ 1.71 (d, 3H), 2.39 (s, 3H), 3.28 (s, 3H), 5.92 (q, 1H), 7.20 (s, 1H), 7.33-7.36 (m, 1H), 7.41-7.46 (m, 1H), 7.67-7.71 (m, 1H), 7.73 (s, 1H).

In a similar manner the following compounds were synthesized:

| Example | Structure | Name | Yield |
|---|---|---|---|
| 6.2 | 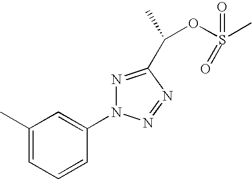 | (S)-1-(2-m-Tolyl-2H-tetrazol-5- yl)-ethyl methanesulfonate | 1.1 g, 100% |
| ¹H NMR | (600 MHz, DMSO-d6) δ 1.81 (d, 3H), 2.44 (s, 3H), 3.29 (s, 3H), 6.18 (d, | | |
| | 1H), 7.43 (d, 1H), 7.54 (t, 1H), 7.88 (d, 1H), 7.91 (s, 1H) | | |
| 6.3 | 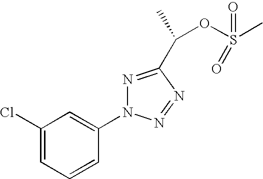 | (S)-1-(2-(3-Chlorophenyl)-2H- tetrazol-5-yl)ethyl methane- sulfonate | 539 mg, 100% |
| ¹H NMR | (600 MHz, DMSO-d6) δ 1.82 (d, 3H), 6.19 (q, 1H), 7.71 (d, 2H), 8.08 (s, | | |
| | 1H), 8.14 (s, 1H) | | |
| 6.4 | 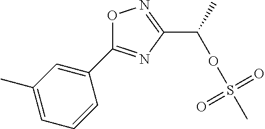 | (S)-1-(5-m-Tolyl-1,2,4-oxadiazol- 3-yl)ethyl methanesulfonate | 312 mg, 100% |

| | |
|---|---|
| ¹H NMR | (600 MHz, DMSO-d6) δ 1.72 (d, 3H), 2.41 (s, 3H), 3.30 (s, 3H), 5.99 (q, |
| | 1H), 7.48-7.56 (m, 2H), 7.92 (d, 1H), 7.95 (s, 1H) |

### Example 7.1

### 4-(4-Methyl-5-thioxo-4,5-dihydro-1H-1,2,4-triazol-3-yl)pyridin-2(1H)-one

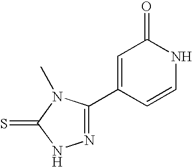

2-Oxo-1,2-dihydropyridine-4-carbohydrazide (5.1 g, 33 mmol) was added to water (50 mL). Isothiocyanatomethane (3.7 g, 50 mmol) was added and the mixture was heated to 50° C. for 4 h. Sodium hydroxide (4.0 g, 99 mmol) in H₂O (30 mL) was added and the mixture to was allowed to stir at 50° C. for 1 h. The mixture was cooled to room temp and HOAc was added until the mixture reached pH 4. The solids were filtered off to give crude title compound (5.5 g, 80%).

¹H NMR (400 MHz, DMSO-d6) δ 3.57 (s, 3H), 6.44-6.48 (m, 1H), 6.70-6.72 (m, 1H), 7.54 (d, 1H), 11.91 (bs, 1H), 14.09 (bs, 1H).

In a similar manner the following compound was synthesized:

| Example | Structure | Name | Yield |
|---|---|---|---|
| 7.2 | 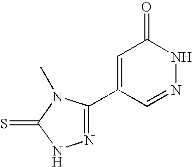 | 5-(4-Methyl-5-thioxo-4,5-dihydro- 1H-1,2,4-triazol-3-yl)pyridazin- 3(2H)-one | 72% 541 mg |

| | |
|---|---|
| ¹H NMR | (600 MHz, DMSO-d6) δ 3.60 (s, 3H), 7.29 (s, 1H), 8.12 (s, 1H), 13.35 (s, |
| | 1H), 14.26 (s, 1H) |

### Example 8.1

### (R)-4-(4-Methyl-5-(1-(2-m-tolyl-2H-tetrazol-5-yl)ethylthio)-4H-1,2,4-triazol-3-yl)pyridin-2(1H)-one

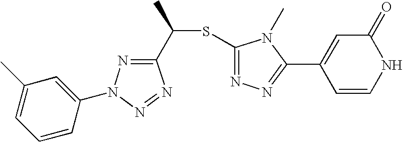

4-(4-Methyl-5-thioxo-4,5-dihydro-1H-1,2,4-triazol-3-yl)pyridin-2(1H)-one (84 mg, 0.40 mmol) and (S)-1-(2-m-tolyl-2H-tetrazol-5-yl)ethyl methanesulfonate (103 mg, 0.36 mmol) was mixed in DMSO (1 mL), N-ethyl-N-isopropylpropan-2-amine (0.13 mL, 0.73 mmol) was added and the mixture was heated to 90° C. for 1 h. The crude compound was purified by preparative HPLC on a XBridge C18 column (10 μm 250×50 ID mm) using a gradient of 10-55% ACN in H₂O/ACN/NH₃ (95:5:0.2) buffer over 30 min with a flow of 100 mL/min Product fractions was freeze dried to give the title compound (72 mg, 50%). The stereochemical purity was determined using Chiral HPLC (Method D), ee: 91.8%.

¹H NMR (400 MHz, DMSO-d6) δ 1.86 (d, 3H), 2.41 (s, 3H), 3.58 (s, 3H), 5.15 (q, 1H), 6.46-6.50 (m, 1H), 6.59-6.62 (m, 1H), 7.38-7.44 (m, 1H), 7.49-7.57 (m, 2H), 7.76-7.83 (m, 2H), 11.86 (bs, 1H).

HRMS (M+H)⁺: calcd 395.1402. found 395.1385.

In a similar manner the following compound was synthesized:

| Example | Structure | Name | Yield |
|---|---|---|---|
| 82 | 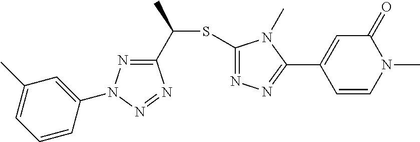 | (R)-1-Methyl-4-(4-methyl-5-(1-(2- m-tolyl-2H-tetrazol-5-yl)ethylthio)- 4H-1,2,4-triazol-3-yl)pyridin- 2(1H)-one | 103 mg, 45% |
| ¹H NMR | The stereochemical purity was determined using Chiral HPLC (Method D), | | |
| and | ee: 91.7%. | | |
| HRMS | (400 MHz, DMSO-d6) δ 1.86 (d, 3H), 2.41 (s, 3H), 3.47 (s, 3H), 3.58 (s, | | |
| | 3H), 5.15 (q, 1H), 6.53 (dd, 1H), 6.68 (d, 1H), 7.39-7.44 (m, 1H), 7.49- | | |
| | 7.55 (m, 1H), 7.77-7.82 (m, 2H), 7.84-7.87 (m, 1H). | | |
| | HRMS (M + H)⁺: calcd 409.1559; found 409.1538 | | |
| 8.3 | 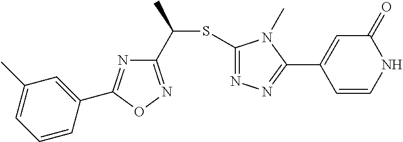 | (R)-4-(4-Methyl-5-(1-(5-m-tolyl- 1,2,4-oxadiazol-3-yl)ethylthio-4H- 1,2,4-triazol-3-yl)pyridin-2(1H)- one | 89 mg, 80% |
| ¹H NMR | The stereochemical purity was determined using Chiral HPLC (Method D), | | |
| and | ee: 92.4%. | | |
| HRMS | (400 MHz, DMSO-d6) δ 1.77 (d, 3H), 2.40 (s, 3H), 3.61 (s, 3H), 4.94 (q, | | |
| | 1H), 6.48-6.53 (m, 1H), 6.61-6.65 (m, 1H), 7.47-7.57 (m, 3H), 7.83- | | |
| | 7.90 (m, 2H). | | |
| | HRMS (M + H)⁺: calcd 395.1290; found 395.1272 | | |
| 8.4 | 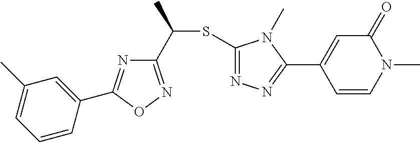 | (R)-1-Methyl-4-(4-methyl-5-(1-(5- m-tolyl-1,2,4-oxadiazol-3- yl)ethylthio)-4H-1,2,4-triazol-3- yl)pyridin-2(1H)-one | 85 mg, 59% |
| ¹H NMR | The stereochemical purity was determined using Chiral HPLC (Method D), | | |
| and | ee: 90.8%. | | |
| HRMS | (400 MHz, DMSO-d6) δ 1.77 (d, 3H), 2.40 (s, 3H), 3.48 (s, 3H), 3.62 (s, | | |
| | 3H), 4.94 (q, 1H), 6.55 (dd, 1H), 6.69-6.70 (m, 1H), 7.47-7.55 (m, 2H), | | |
| | 7.84-7.88 (m, 3H). | | |
| | HRMS (M + H)⁺: calcd 409.1447; found 409.1432 | | |
| 8.5 | 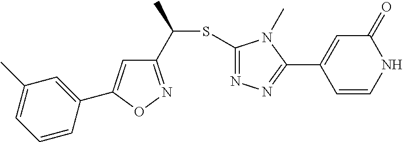 | (R)-4-(4-Methyl-5-(1-(5-m- tolylisoxazol-3-yl)ethylthio)-4H- 1,2,4-triazol-3-yl)pyridin-2(1H)- one | 126 mg, 53% |
| ¹H NMR | The stereochemical purity was determined using Chiral HPLC (Method G), | | |
| and | ee: 89.8% | | |
| HRMS | (600 MHz, DMSO-d6) δ 1.74 (d, 3H), 2.38 (s, 3H), 3.62 (s, 3H), 4.88 (q, | | |
| | 1H), 6.48-6.51 (m, 1H), 6.62-6.64 (m, 1H), 7.07-7.10 (m, 1H), 7.31- | | |
| | 7.34 (m, 1H), 7.40-7.44 (m, 1H), 7.51-7.55 (m, 1H), 7.62-7.65 (m, 1H), | | |
| | 7.66-7.68 (m, 1H). | | |
| | HRMS (M + H)⁺: calcd 394.1338; found 394.1331 | | |
| 8.6 | 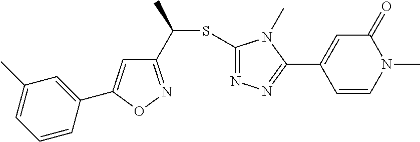 | (R)-1-Methyl-4-(4-methyl-5-(1-(5- m-tolylisoxazol-3-yl)ethylthio)-4H- 1,2,4-triazol-3-yl)pyridin-2(1H)- one | 135 mg, 60% |
| ¹H NMR | The stereochemical purity was determined using Chiral HPLC (Method C), | | |
| and | cc: 85.6%. | | |
| HRMS | (400 MHz, DMSO-d6) δ 1.74 (d, 3H), 2.38 (s, 3H), 3.47 (s, 3H), 3.62 (s, | | |
| | 3H), 4.88 (q, 1H), 6.53-6.57 (m, 1H), 6.69-6.72 (m, 1H), 7.09 (s, 1H), | | |
| | 7.30-7.35 (m, 1H), 7.39-7.45 (m, 1H), 7.61-7.69 (m, 2H), 7.85 (d, 1H). | | |
| | HRMS (M + H)⁺: calcd 408.1494; found 408.1486 | | |
| 8.7 | 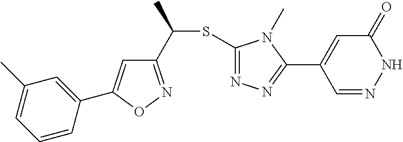 | (R)-5-(4-Methy1-5-(1-(5-m- tolylisoxazol-3-yl)ethylthio)-4H- 1,2,4-triazol-3-yl)pyridazin-3(2H)- one | 134 mg, 60% |
| ¹H NMR | The stereochemical purity was determined using Chiral HPLC (Method A), | | |
| and | ee: 80.9%. | | |
| HRMS | (400 MHz, DMSO-d6) δ 1.73 (d, 3H), 2.36 (s, 3H), 3.67 (s, 3H), 4.91 (q, | | |
| | 1H), 7.09 (s, 1H), 7.19-7.21 (m, 1H), 7.29-7.33 (m, 1H), 7.38-7.43 (m, | | |
| | 1H), 7.60- 7.67 (m, 2H), 8.20-8.22 (m, 1H). | | |
| | HRMS (M + H)⁺: calcd 395.1290; found 395.1271 | | |
| 8.8 | 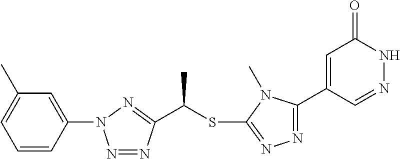 | (R)-5-(4-Methyl-5-(1-(2-m-tolyl- 2H-tetrazol-5-yl)ethylthio)-4H- 1,2,4-triazol-3-yl)pyridazin-3(2H)- one | 181 mg, 59% |
| ¹H NMR | The stereochemical purity was determined using Chiral HPLC (Method F), | | |
| and | ee: 79.8%. | | |
| HRMS | (600 MHz, DMSO-d6) δ 1.85 (d, 3H), 2.40 (s, 3H), 3.64 (s, 3H), 5.17 (d, | | |
| | 1H), 7.17 (s, 1H), 7.39 (d, 1H), 7.50 (t, 1H), 7.79 (s, 2H), 8.19 (s, 1H), 13.31 | | |
| | (s, 1H). | | |
| | HRMS (M + H)⁺: calcd 396.1355; found 396.1380 | | |
| 8.9 | 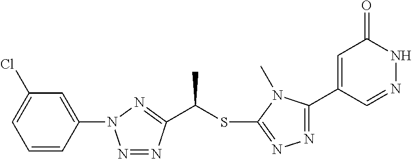 | (R)-5-(5-(1-(2-(3-Chlorophenyl)- 2H-tetrazol-5-yl)ethylthio)-4- methyl-4H-1,2,4-triazol-3- yl)pyridazin-3(2H)-one | 180 mg, 55% |
| ¹H NMR | The stereochemical purity was determined using Chiral HPLC (Method E), | | |
| and | ee: 71.8%. | | |
| HRMS | (600 MHz, DMSO-d6) δ 1.85 (d, 3H), 3.65 (s, 3H), 5.18 (d, 1H), 7.19 (s, | | |
| | 1H), 7.67 (s, 2H), 8.00 (s, 2H), 8.19 (s, 1H), 13.31 (s, 1H). | | |
| | HRMS (M + H)⁺: calcd 416.0809; found 416.0811 | | |
| 8.10 | 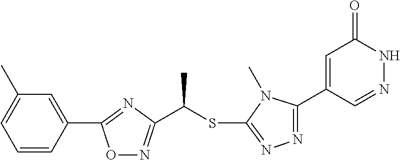 | (R)-5-(4-Methyl-5-(1-(5-m-tolyl- 1,2,4-oxadiazol-3-yl)ethylthio)-4H- 1,2,4-triazol-3-yl)pyridazin-3(2H)- one | 225 mg, 75% |

| | |
|---|---|
| ¹H NMR | The stereochemical purity was determined using Chiral HPLC (Method B), |
| and | ee: 89.5%. |
| HRMS | (600 MHz, DMSO-d6) δ 1.76 (d, 3H), 2.38 (s, 3H), 3.66 (s, 3H), 4.95 (q, |
| | 1H), 7.17 (s, 1H), 7.50 (dt, 2H), 7.85 (d, 2H), 8.21 (d, 1H), 13.31 (s, 1H). |
| | HRMS (M + H)⁺: calcd 396.1243; found 396.1254 |

### Biological Evaluation

Functional Assessment of mGluR5 Antagonism in Cell Lines Expressing mGluR5D

The properties of the compounds of the invention can be analyzed using standard assays for pharmacological activity. Examples of glutamate receptor assays are well known in the art as described in for example Aramori et al., Neuron 8:757 (1992), Tanabe et al., Neuron 8:169 (1992), Miller et al., J. Neuroscience 15: 6103 (1995), Balazs, et al., J. Neurochemistry 69:151 (1997). The methodology described in these publications is incorporated herein by reference. Conveniently, the compounds of the invention can be studied by means of an assay (FLIPR) that measures the mobilization of intracellular calcium, [Ca²⁺]ᵢ in cells expressing mGluR5 or another assay (IP3) that measures inositol phosphate accumulation.

### FLIPR Assay

Cells expressing human mGluR5d as described in WO97/05252 cultured in a mixture of high glucose DMEM with Glutamax (31966-021) (500 mL), 10% dialyzed fetal bovine serum (Hyclone #SH30079.03)(56 mL), 200 ug/mL Hygromycin B (Invitrogen 45-0430, 50 mg/mL) (2.2 mL), 200 ug/mL Zeocin (Invitrogen #R250-01; 100 mg/mL) (1.1 mL) are seeded at a density of 15,000 cells per well on a black poly-D Lysin coated 384-plate with clear bottom (BD Biocoat 356663). Cells are allowed to adhere over night before experiments. All assays are done in a buffer containing 146 mM NaCl, 5 mM KCl, 1 mM MgCl₂, 1 mM CaCl₂, 20 mM HEPES, 1 mg/mL glucose and 1 mg/mL BSA Fraction IV (pH 7.4). Cell cultures in the 384-well plates are loaded for 60 minutes in the above mentioned buffer containing 3 μM of the acetoxymethyl ester form of the fluorescent calcium indicator fluo-4 (Molecular Probes, Eugene, Oreg.) in 0.025% pluronic acid (a proprietary, non-ionic surfactant polyol—CAS Number 9003-11-6). Following the loading period, the fluo-4 buffer is removed and the cellplate is washed three times with fresh assay buffer. FLIPR experiments are done using a laser setting of 1.0 W and 0.4 second CCD camera shutter speed with excitation and emission wavelengths of 488 nm and 562 nm, respectively.

Each experiment is initiated with 20 μl of buffer present in each well of the cell plate when 7.5 μl from the antagonist plate is added. After 30 minutes incubation in the dark at 25° C., 20 μL from the agonist plate is added to the wells. The fluorescence signal is sampled 60 times at 1 second intervals followed by 20 samples at 6 seconds intervals immediately after the agonist addition. Responses are measured as the difference between the peak height of the response to agonist, less the background fluorescence within the sample period. IC₅₀ determinations are made using a linear least squares fitting program.

### IP3 Assay

An additional functional assay for mGluR5d is described in WO97/05252 and is based on phosphatidylinositol turnover. Receptor activation stimulates phospholipase C activity and leads to increased formation of inositol 1,4,5,triphosphate (IP₃). GHEK stably expressing the human mGluR5d are seeded onto 24 well poly-L-lysine coated plates at 40×10⁴ cells/well in media containing 1 μCi/well [3H] myo-inositol. Cells are incubated overnight (16 h), then washed three times and incubated for 1 h at 37° C. in HEPES buffered saline (146 mM NaCl, 4.2 mM KCl, 0.5 mM MgCl₂, 0.1% glucose, 20 mM HEPES, pH 7.4) supplemented with 1 unit/mL glutamate pyruvate transaminase and 2 mM pyruvate. Cells are washed once in HEPES buffered saline and pre-incubated for 10 mM in HEPES buffered saline containing 10 mM LiCl. Compounds are incubated in duplicate at 37° C. for 15 mM, then either glutamate (80 μM) or DHPG (30 μM) is added and incubated for an additional 30 min. The reaction is terminated by the addition of 0.5 mL perchloric acid (5%) on ice, with incubation at 4° C. for at least 30 min. Samples are collected in 15 mL polyproplylene tubes and inositol phosphates are separated using ion-exchange resin (Dowex AG1-X8 formate form, 200-400 mesh, BIORAD) columns. Inositol phosphate separation is done by first eluting glycero phosphatidyl inositol with 8 mL 30 mM ammonium formate. Next, total inositol phosphates is eluted with 8 mL 700 mM ammonium formate/100 mM formic acid and collected in scintillation vials. This eluate is then mixed with 8 mL of scintillant and [3H] inositol incorporation is determined by scintillation counting. The dpm counts from the duplicate samples are plotted and IC₅₀ determinations are generated using a linear least squares fitting program.

### ABBREVIATIONS

### BSA Bovine Serum Albumin

### CCD Charge Coupled Device

### CRC Concentration Response Curve

### DHPG 3,5-Dihydroxyphenylglycine

### DPM Disintegrations per Minute

### EDTA Ethylene Diamine Tetraacetic Acid

### FLIPR Fluorometric Imaging Plate Reader

GHEK GLAST-transfected HEK293 cells
GLAST Glutamate/aspartate transporter
HEPES 4-(2-Hydroxyethyl)-1-piperazineethanesulfonic acid (buffer)
IP₃ Inositol triphosphate

Generally, the compounds were active in the FLIPR assay above with IC₅₀ values less than 10 000 nM. In one aspect of the invention, the IC₅₀ value is less than 1 000 nM. In a further aspect of the invention, the IC₅₀ value is less than 100 nM.

### Determination of Brain to Plasma Ratio in Rat

Brain to plasma ratios are estimated in female Sprague Dawley rats. The compound is dissolved in water or another appropriate vehicle. For determination of brain to plasma ratio the compound is administrated as a subcutaneous, or an intravenous bolus injection, or an intravenous infusion, or an oral administration. At a predetermined time point after the administration a blood sample is taken with cardiac puncture. The rat is terminated by cutting the heart open, and the brain is immediately retained. The blood samples are collected in heparinized tubes and centrifuged within 30 minutes, in order to separate the plasma from the blood cells. The plasma is transferred to 96-well plates and stored at −20° C. until analysis. The brains are divided in half, and each half is placed in a pre-tarred tube and stored at −20° C. until analysis. Prior to the analysis, the brain samples are thawed and 3 mL/g brain tissue of distilled water is added to the tubes. The brain samples are sonicated in an ice bath until the samples are homogenized. Both brain and plasma samples are precipitated with acetonitrile. After centrifugation, the supernatant is diluted with 0.2% formic acid. Analysis is performed on a short reversed-phase HPLC column with rapid gradient elution and MSMS detection using a triple quadrupole instrument with electrospray ionisation and Selected Reaction Monitoring (SRM) acquisition. Liquid-liquid extraction may be used as an alternative sample clean-up. The samples are extracted, by shaking, to an organic solvent after addition of a suitable buffer. An aliquot of the organic layer is transferred to a new vial and evaporated to dryness under a stream of nitrogen. After reconstitution of the residuals the samples are ready for injection onto the HPLC column.

Generally, the compounds according to the present invention are peripherally restricted with a drug in brain over drug in plasma ratio in the rat of <0.5. In one embodiment, the ratio is less than 0.15.

### Determination of In Vitro Stability

Rat liver microsomes are prepared from Sprague-Dawley rats liver samples. Human liver microsomes are either prepared from human liver samples or acquired from BD Gentest. The compounds are incubated at 37° C. at a total microsome protein concentration of 0.5 mg/mL in a 0.1 mol/L potassium phosphate buffer at pH 7.4, in the presence of the cofactor, NADPH (1.0 mmol/L). The initial concentration of compound is 1.0 μmol/L. Samples are taken for analysis at 5 time points, 0, 7, 15, 20 and 30 minutes after the start of the incubation. The enzymatic activity in the collected sample is immediately stopped by adding a 3.5 times volume of acetonitrile. The concentration of compound remaining in each of the collected samples is determined by means of LC-MS. The elimination rate constant (k) of the mGluR5 inhibitor is calculated as the slope of the plot of In[mGluR5 inhibitor] against incubation time (minutes). The elimination rate constant is then used to calculate the half-life (T ½) of the mGluR5 inhibitor, which is subsequently used to calculate the intrinsic clearance (CLint) of the mGluR5 inhibitor in liver microsomes as: CLint.=(ln2×incubation volume)/(T ½×protein concentration)=μL/min/mg

### Screening for Compounds Active Against TLESR

Adult Labrador retrievers of both genders, trained to stand in a Pavlov sling, are used. Mucosa-to-skin esophagostomies are formed and the dogs are allowed to recover completely before any experiments are done.

### Motility Measurement

In brief, after fasting for approximately 17 h with free supply of water, a multilumen sleeve/sidehole assembly (Dentsleeve, Adelaide, South Australia) is introduced through the esophagostomy to measure gastric, lower esophageal sphincter (LES) and esophageal pressures. The assembly is perfused with water using a low-compliance manometric perfusion pump (Dentsleeve, Adelaide, South Australia). An air-perfused tube is passed in the oral direction to measure swallows, and an antimony electrode monitored pH, 3 cm above the LES. All signals are amplified and acquired on a personal computer at 10 Hz.

When a baseline measurement free from fasting gastric/LES phase III motor activity has been obtained, placebo (0.9% NaCl) or test compound is administered intravenously (i.v., 0.5 mL/kg) in a foreleg vein. Ten min after i.v. administration, a nutrient meal (10% peptone, 5% D-glucose, 5% Intralipid, pH 3.0) is infused into the stomach through the central lumen of the assembly at 100 mL/min to a final volume of 30 mL/kg. The infusion of the nutrient meal is followed by air infusion at a rate of 500 mL/min until an intragastric pressure of 10±1 mmHg is obtained. The pressure is then maintained at this level throughout the experiment using the infusion pump for further air infusion or for venting air from the stomach. The experimental time from start of nutrient infusion to end of air insufflation is 45 min. The procedure has been validated as a reliable means of triggering TLESRs.

TLESRs is defined as a decrease in lower esophageal sphincter pressure (with reference to intragastric pressure) at a rate of >1 mmHg/s. The relaxation should not be preceded by a pharyngeal signal≦2 s before its onset in which case the relaxation is classified as swallow-induced. The pressure difference between the LES and the stomach should be less than 2 mmHg, and the duration of the complete relaxation longer than 1 s.

Specimen results are shown in the following Table:

| | | Brain/Plasma Ratio |
|---|---|---|
| Example | FLIPR hmGluR5d (nM) | of compound in Rat |
| 8.1 | 19 | <0.01 |
| 8.2 | 87 | 0.10 |
| 8.3 | 24 | 0.09 |
| 8.4 | 30 | 0.16 |
| 8.5 | 146 | <0.01 |
| 8.6 | 380 | 0.02 |
| 8.7 | 19 | 0.26 |
| 8.8 | 18 | <0.010 |
| 8.9 | 18 | <0.015 |
| 8.10 | 41 | 0.040 |

1. A compound of formula (I) 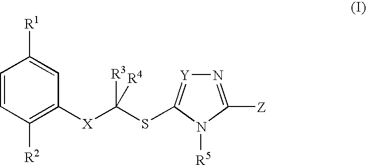
wherein
R¹ is methyl, halogen or cyano;
R² is hydrogen or fluoro;
R³ is hydrogen, C₁-C₃ alkyl or cyclopropyl;
R⁴ is hydrogen, C₁-C₃ alkyl or cyclopropyl;
R⁵ is C₁-C₃ alkyl or cyclopropyl;
X is 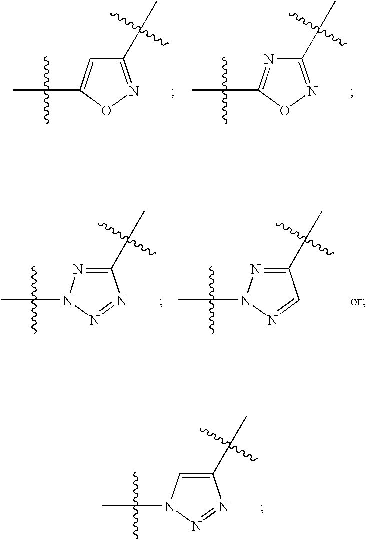
Y is C or N;
and Z is 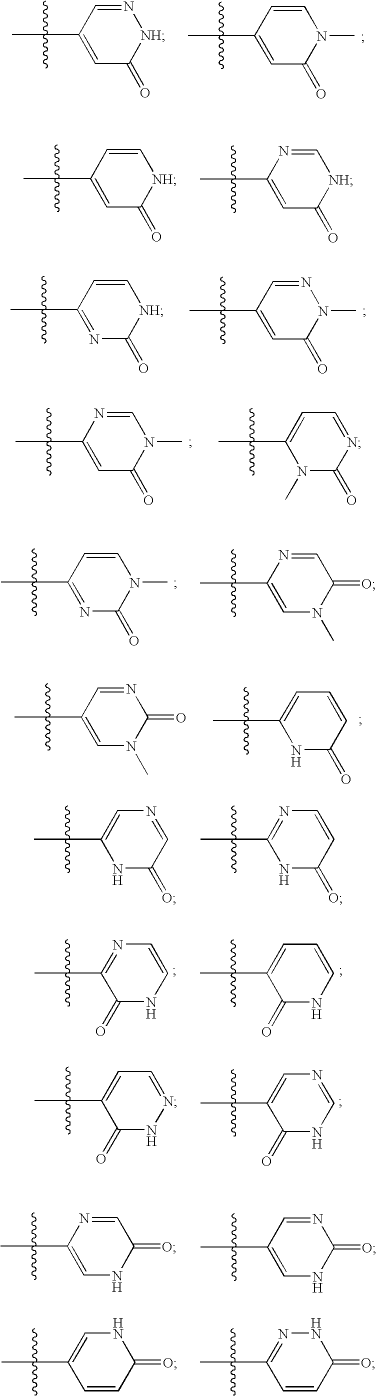 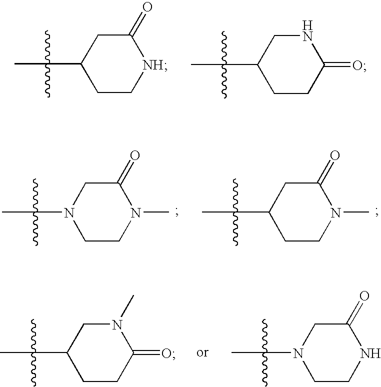
or a pharmaceutically acceptable salt, hydrate, isoform, tautomer or enantiomer thereof.

2. A compound according to claim 1, wherein R¹ is chloro or methyl and/or R² is hydrogen.

3. A compound according to claim 1, wherein R³ is C₁-C₃ alkyl or cyclopropyl and R⁴ is hydrogen.

4. A compound according to claim 1, wherein the sum of the number of carbon atoms in the substituents R³ and R⁴ is 2 or less.

5. A compound according to claim 1, wherein R⁵ is methyl.

6. A compound according to claim 1, wherein X is 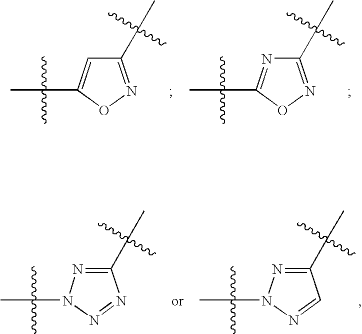
and/or Y is N.

7. A compound according to claim 1, wherein Z

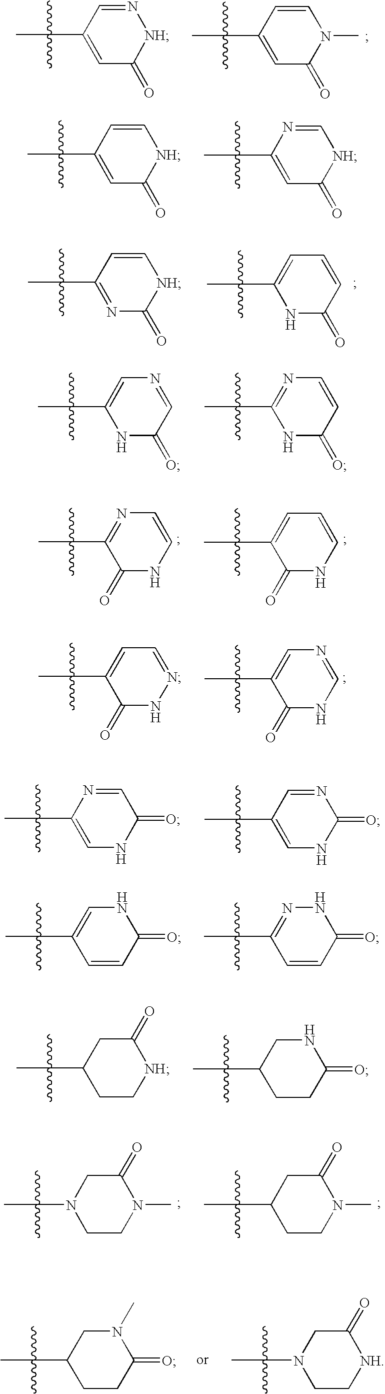

8. A compound according to claim 7, wherein Z is

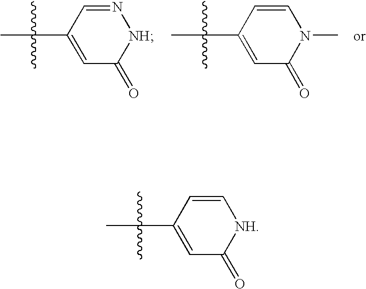

9. A compound according to claim 1, wherein the orientation of X is such that the compound of formula (I) has a structure selected from

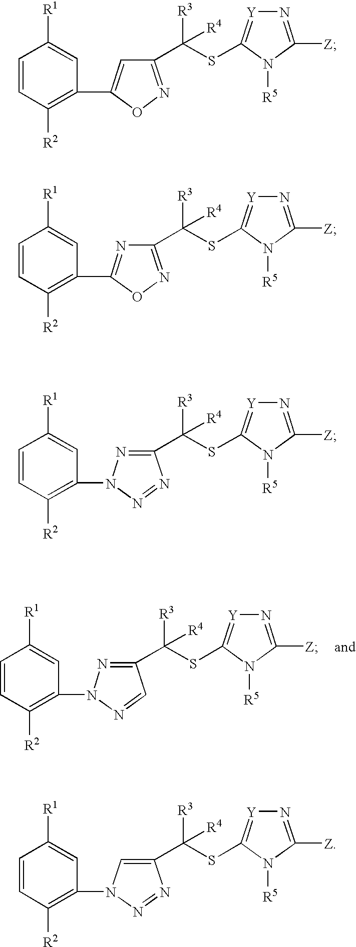

10. A compound according to claim 1, wherein
R¹ is methyl or chloro;
R² is hydrogen;
R³ is methyl;
R⁴ is hydrogen;
R⁵ is methyl;
X is 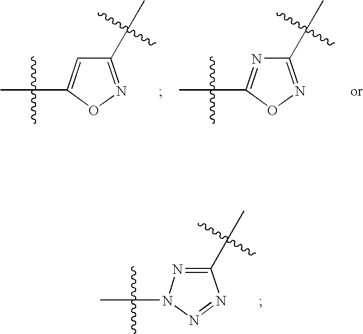
Z is 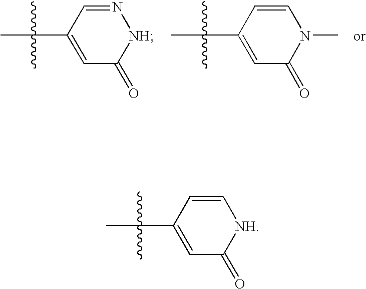
as well as pharmaceutically acceptable salts, hydrates, isoforms, tautomers and/or enantiomers thereof.

11. A compound according to claim 1, wherein R⁴ is hydrogen and R³ is C₁-C₃ alkyl or cyclopropyl and the stereochemistry of the compound of formula (I) is such that R³ projects out of the plane and R⁴ projects into the plane.

12. A compound according to claim 11, wherein the stereochemistry of the compound of formula (I) is such that the compound of formula (I) has the structure

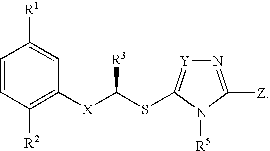

13. A compound according to claim 1 selected from
(R)-4-(4-methyl-5-(1-(2-m-tolyl-2H-tetrazol-5-yl)ethylthio)-4H-1,2,4-triazol-3-yl)pyridin-2(1H)-one;
(R)-1-methyl-4-(4-methyl-5-(1-(2-m-tolyl-2H-tetrazol-5-yl)ethylthio)-4H-1,2,4-triazol-3-yl)pyridin-2(1H)-one;
(R)-4-(4-methyl-5-(1-(5-m-tolyl-1,2,4-oxadiazol-3-yl)ethylthio)-4H-1,2,4-triazol-3-yl)pyridin-2(1H)-one;
(R)-1-methyl-4-(4-methyl-5-(1-(5-m-tolyl-1,2,4-oxadiazol-3-yl)ethylthio)-4H-1,2,4-triazol-3-yl)pyridin-2(1H)-one;
(R)-4-(4-methyl-5-(1-(5-m-tolylisoxazol-3-yl)ethylthio)-4,4-1,2,4-triazol-3-yl)pyridin-2(1H)-one;
(R)-1-methyl-4-(4-methyl-5-(1-(5-m-tolylisoxazol-3-yl)ethylthio)-4H-1,2,4-triazol-3-yl)pyridin-2(1H)-one;
(R)-5-(4-methyl-5-(1-(5-m-tolylisoxazol-3-yl)ethylthio)-4H-1,2,4-triazol-3-yl)pyridazin-3(2H)-one;
(R)-5-(4-methyl-5-(1-(2-m-tolyl-2H-tetrazol-5-yl)ethylthio)-4H-1,2,4-triazol-3-yl)pyridazin-3(2H)-one;
(R)-5-(5-(1-(2-(3-chlorophenyl)-2H-tetrazol-5-yl)ethylthio)-4-methyl-4H-1,2,4-triazol-3-yl)pyridazin-3(2H)-one; and
(R)-5-(4-methyl-5-(1-(5-m-tolyl-1,2,4-oxadiazol-3-yl)ethylthio)-4H-1,2,4-triazol-3-yl)pyridazin-3(2H)-one;
as well as pharmaceutically acceptable salts, hydrates, isoforms, tautomers and/or enantiomers thereof.

14. A compound according to claim 1 for use in therapy.

15. A pharmaceutical composition comprising a compound according to claim 1 as an active ingredient, together with a pharmacologically and pharmaceutically acceptable carrier.

16. A method for the inhibition of transient lower esophageal sphincter relaxations wherein an effective amount of a compound according to claim 1 is administered to a subject in need of such inhibition.

17. A method for the treatment or prevention of gastroesophageal reflux disease, wherein an effective amount of the compound according to claim 1 is administered to a subject in need of such treatment or prevention.

18. A method for the treatment or prevention of pain, wherein an effective amount of the compound according to claim 1 is administered to a subject in need of such treatment or prevention.

19. A method for the treatment or prevention of anxiety, wherein an effective amount of the compound according to claim 1 is administered to a subject in need of such treatment or prevention.

20. A method for the treatment or prevention of irritable bowel syndrome (IBS), wherein an effective amount of the compound according to claim 1 is administered to a subject in need of such treatment or prevention.

21. A combination comprising (i) at least one compound according to claim 1 and (ii) at least one acid secretion inhibiting agent.

22. A combination according to claim 21 wherein the acid secretion inhibiting agent is selected from cimetidine, ranitidine, omeprazole, esomeprazole, lansoprazole, pantoprazole, rabeprazole or leminoprazole.

23. A compound selected from
(E)-ethyl 2-amino-2-(3-methylbenzoyloxyimino)acetate;
ethyl 5-m-tolyl-1,2,4-oxadiazole-3-carboxylate;
1-(2-m-tolyl-2H-tetrazol-5-yl)ethanone;
1-(5-m-tolylisoxazol-3-yl)ethanone;
1-(5-m-tolyl-1,2,4-oxadiazol-3-yl)ethanone;
(S)-1-(5-m-tolylisoxazol-3-yl)ethanol;
(S)-1-(5-m-tolyl-1,2,4-oxadiazol-3-yl)ethanol;
(S)-1-(2-m-tolyl-2H-tetrazol-5-yl)ethanol;
(S)-1-(5-m-tolylisoxazol-3-yl)ethyl methanesulfonate;
(S)-1-(2-m-tolyl-2H-tetrazol-5-yl)ethyl methanesulfonate;
(S)-1-(2-(3-chlorophenyl)-2H-tetrazol-5-yl)ethyl methanesulfonate;
(S)-1-(5-m-tolyl-1,2,4-oxadiazol-3-yl)ethyl methanesulfonate;
4-(4-methyl-5-thioxo-4,5-dihydro-1H-1,2,4-triazol-3-yl)pyridin-2(1H)-one; and
5-(4-methyl-5-thioxo-4,5-dihydro-1H-1,2,4-triazol-3-yl)pyridazin-3(2H)-one.
